(19) United States
(12) Patent Application Publication
(10) Pub. No.: US 2014/0342022 A1
(43) Pub. Date: Nov. 20, 2014

(54) Natural Product Against Fatigue

(71) Applicant: Hanan Polansky, Rochester, NY (US)

(72) Inventor: Hanan Polansky, Rochester, NY (US)

(21) Appl. No.: 13/893,564

(22) Filed: May 14, 2013

(51) Int. Cl. A61K 36/82; A61K 33/04; A61K 36/54; A61K 31/352; A61K 36/484

(52) U.S. Cl. CPC A61K 36/82; A61K 31/352; A61K 36/484; A61K 36/54; A61K 33/04
USPC 424/702

(57) ABSTRACT

Gene-Eden-VIR is a natural product that safely and effectively decreases the level of fatigue in treated individuals.

### BACKGROUND OF THE INVENTION

Excessive, or pathologic fatigue is a common complaint by both patients and healthy people (Huskisson 2007, Havard 1985). Clinical signs of excessive fatigue include lack of energy, exhaustion after relatively little exertion, sleepiness, inability to complete simple activities of daily living, difficulty in simple movements, stiffness, lack of motivation, problems concentrating, problem remembering, problems making decisions, and, in general, reduced ability to perform physical and mental tasks.

Excessive fatigue is a common symptom of many diseases (Cho 2012, including acute infections, malignancy, cardiac diseases, inflammation, medications, or disorders such as psychiatric, neurological, endocrine, rheumatologic, genetic, metabolic, and electrolyte (Appels and Mulder 1989, Judge 2000, Theobald 2004, Cho 2012). Other diseases include multiple sclerosis, stroke, traumatic brain injury, other neurologic conditions, cardiovascular disease, systemic lupus erythematosus, cancer, depression, and sleeping disorders. In many of these diseases, fatigue is a typical symptom, and is sometimes associated with markers of disease activity, including cytokines and complement activity. Fatigue may also be associated with psychological factors, such as fears, stress, anxieties, and obsessive and compulsive thoughts. Studies showed a lack of correlation between self-reported fatigue and results of physical performance tests.

There are many behavioral modification methods used to target fatigue, such as diets and physical activity. However, there are no drugs specifically approved for decreasing the level of fatigue.

There are also some dietary supplements, which claim to decrease the level of fatigue. In recent years, there has been an increase in the demand for natural medicine, and there is growing evidence that they are both effective and safe (Lakhan and Vieira 2010). The 2007 National Health Interview Survey (NHIS) found that approximately 40 percent of adult Americans used Complementary and Alternative (CAM) medicine in 2007 (Barnes 2007). Another recent study showed that CAM therapies are increasingly integrated into health care practices. According to the American Hospital Association's Annual Survey of Hospitals, the percentage of hospitals offering natural therapies has increased from 7.9 percent in 1998 to 19.8 percent in 2006 (Johnson 2012). However, unlike conventional medicines, natural remedies are not considered as drugs, and therefore, are not regulated as such by the FDA (FDA 2004). As a result, most natural products available today lack clinical proof for their efficacy, specifically, the kind required by the FDA for new drug approval.

### BRIEF SUMMARY OF THE INVENTION

Gene-Eden-VIR is a natural product that safely and effectively decreases the level of fatigue in treated individuals.

### DETAILED DESCRIPTION OF THE INVENTION

Gene-Eden-VIR is a natural product designed to control the viral copy number in latently infected individuals, that is, asymptomatic individuals.

Gene-Eden-VIR was formulated by analyzing the text in thousands of scientific papers with a proprietary psycholinguistic-based, data-mining program, called Computer Intuition. The research objective was to identify natural ingredients that have a strong antiviral effect against the most common viruses. In this process, the computer analyzed more than 50,000 papers. At the end of this stage, the computer assisted in selecting five ingredients: Green Tea Extract, Quercetin, Licorice Extract, Cinnamomum Extract, and Selenium.

A manual search on these five ingredients supported the Computer Intuition analysis. The search identified a few studies that directly measured the antiviral effect of these ingredients. For instance, some studies showed that catechins, found in green tea, are effective against viruses such as Epstein-Barr Virus (EBV), Herpes Simplex Virus (HSV), Hepatitis Virus B (HVB), and other viruses (Singal 2005, Lyu 2005, Chang 2003, Lin 2000). Other studies showed that quercetin inhibits EBV-EA activation in latently infected cells (Iwase 2001, Ozcelik 2006, Arena 2008). Some studies showed that glycyrrhizin and glycyrrhizic acid, found in licorice, have an antiviral effect (Sekizawa 2001, Kapadia 2002, Lin 2003, Fiore 2008). A few studies showed that the active compounds in cinnamon: cinnamaldehyde, terpenoids, eugenol, and ethyl cinnamate, have a strong antiviral effect (Benencia 2000, Tragoolpua 2007, Orihara 2008). Finally, some studies reported that selenium has an antiviral effect (Jian 2003, Wojtowicz 2004, Schrauzer 2008).

After selecting the five ingredients, the developers used the Computer Intuition program again to analyze the thousands of papers published on these five ingredients. The following table lists the number of scientific papers published on each ingredient according to PubMed as of Sep. 1, 2009.

TABLE 1
Scientific papers per ingredient according to PubMed

| Ingredient | Number of Scientific Papers |
|---|---|
| Green Tea Extract | 3,413 |
| Quercetin | 6,753 |
| Licorice Extract | 2,215 |
| Cinnamomum Extract | 913 |
| Selenium | 2,004 |
| Total | 15,298 |

At the end of this stage, the developers determined the final formula of Gene-Eden-VIR: Quercetin 100 mg, Green Tea Extract 150 mg, Cinnamon Extract 50 mg, Selenium 100 mcg, and Licorice Extract 25 mg. Gene-Eden-VIR was introduced in the marketplace at the end of 2009.

The current invention centers on the fact that Gene-Eden-VIR decreases the levels of fatigue in treated individuals.

### EXAMPLES

The following section reports the results of a clinical study that proved that Gene-Eden-VIR decreases the level of fatigue in treated individuals.

Methods

Treatment

Participants used 1, 2, 3, or 4 capsules of Gene-Eden-VIR per day. The duration of treatment ranged from 2 to 54 weeks.

Questionnaire

We used a self-developed questionnaire called the Natural Origin Treatment Clinical Questionnaire (NotCiq). The following section presents the questionnaire.

Natural Origin Treatment Clinical Questionnaire (NotCiq)

A. General Health Information

A.1 How many weeks/months have you been taking Gene-Eden? Please indicate the number of weeks/months you've been taking Gene-Eden ______.

A1.2 When was the last time you used Gene-Eden? Date: ______

A.2 Have you been taking Gene-Eden continually or from time to time? ______.

A.3 How many capsules are you taking per day? ______

A.4A Why are you taking Gene-Eden? ______ A.4B What symptoms do you have? ______ A.5 Before you've started taking Gene-Eden, how would you describe your health, in general, on a scale from 1 to 7?

| | | | | | | | | |
|---|---|---|---|---|---|---|---|---|
| Very poor health | 1 | 2 | 3 | 4 | 5 | 6 | 7 | Perfect health |

(Level of weakness, fever, pain, fatigue, sensitivity, nausea, headache, high blood pressure, skin problems, allergies, infections etc.)?

Since taking Gene-Eden, how would you describe your health, on a scale from 1 to 7?

| | | | | | | | | |
|---|---|---|---|---|---|---|---|---|
| Very poor Health | 1 | 2 | 3 | 4 | 5 | 6 | 7 | Perfect health |

A.6 Do you have any side effects from taking Gene-Eden ______

A.7 Do you experience any unexpected health problems since you've started taking Gene-Eden? ______

B. Symptoms

Lastly, I will ask you about your symptoms.

Symptoms severity

B.1 Before taking Gene-Eden, how severe were your ______ (use symptoms from A4 above), on a scale from 1 to 7?

| | | | | | | | | |
|---|---|---|---|---|---|---|---|---|
| Very Severe | 1 | 2 | 3 | 4 | 5 | 6 | 7 | Unnoticeable |

Since taking Gene-Eden, how severe are your ______ (use symptoms from above), on a scale from 1 to 7?

| | | | | | | | | |
|---|---|---|---|---|---|---|---|---|
| Very Severe | 1 | 2 | 3 | 4 | 5 | 6 | 7 | Unnoticeable |

B.2 Before taking Gene-Eden, to what extent did the ______ (use symptoms from above) interfere with daily life, on a scale from 1 to 7?

| | | | | | | | | |
|---|---|---|---|---|---|---|---|---|
| Extremely Interfered | 1 | 2 | 3 | 4 | 5 | 6 | 7 | Not at all |

Since taking Gene-Eden, to what extent do the symptoms interfere, on a scale from 1 to 7?

| | | | | | | | | |
|---|---|---|---|---|---|---|---|---|
| Extremely Interfere | 1 | 2 | 3 | 4 | 5 | 6 | 7 | Not at all |

Symptoms Duration

B.3 Before taking Gene-Eden, how long did the ______ (use symptoms from above) last, on a scale from 1 to 7?

| | | | | | | | | |
|---|---|---|---|---|---|---|---|---|
| All the time | 1 | 2 | 3 | 4 | 5 | 6 | 7 | Not at all |

Since taking Gene-Eden, how long does it last on a scale from 1 to 7?

| | | | | | | | | |
|---|---|---|---|---|---|---|---|---|
| All the time | 1 | 2 | 3 | 4 | 5 | 6 | 7 | Not at all |

B.4 Before taking Gene-Eden, how long lasted a relief of ______ (use symptoms from above) on a scale from 1 to 7?

| | | | | | | | | |
|---|---|---|---|---|---|---|---|---|
| No relief | 1 | 2 | 3 | 4 | 5 | 6 | 7 | Constant relief |

And now, since taking Gene-Eden, how long does it last on a scale from 1 to 7?

| | | | | | | | | |
|---|---|---|---|---|---|---|---|---|
| No relief | 1 | 2 | 3 | 4 | 5 | 6 | 7 | Constant relief |

Symptoms Frequency

B.5 Before taking Gene-Eden, how often (many times a month, a week, a day etc.) did the ______ (use symptoms from above) appear on a scale from 1 to 7?

| | | | | | | | | |
|---|---|---|---|---|---|---|---|---|
| Frequently appeared | 1 | 2 | 3 | 4 | 5 | 6 | 7 | Never appeared |

And now, how often do the symptoms appear?

| | | | | | | | | |
|---|---|---|---|---|---|---|---|---|
| Frequently appear | 1 | 2 | 3 | 4 | 5 | 6 | 7 | Never appear |

C. Physical Tasks

Now I'm going to ask you questions about your physical strength.

C.1 Before you've started taking Gene-Eden, how strong were you physically, on a scale from 1 to 7?

| | | | | | | | | |
|---|---|---|---|---|---|---|---|---|
| Very Weak | 1 | 2 | 3 | 4 | 5 | 6 | 7 | Very Strong |

Since taking Gene-Eden, how strong are you, on a scale from 1 to 7?

| | | | | | | | | |
|---|---|---|---|---|---|---|---|---|
| Very Weak | 1 | 2 | 3 | 4 | 5 | 6 | 7 | Very Strong |

C.2 Before taking Gene-Eden, how would you describe your ability to do your job (at work) on a scale from 1 to 7?

| | | | | | | | | |
|---|---|---|---|---|---|---|---|---|
| Completely Unable | 1 | 2 | 3 | 4 | 5 | 6 | 7 | Completely Able |

Since taking Gene-Eden, how would you describe your ability to do you job, on a scale from 1 to 7?

| | | | | | | | | |
|---|---|---|---|---|---|---|---|---|
| Completely Unable | 1 | 2 | 3 | 4 | 5 | 6 | 7 | Completely Able |

C.3 Before taking Gene-Eden, how would you describe your ability to exercise (sports, climb stairs, walk several blocks) on a scale from 1 to 7?

| | | | | | | | | |
|---|---|---|---|---|---|---|---|---|
| Completely Unable | 1 | 2 | 3 | 4 | 5 | 6 | 7 | Completely Able |

Since taking Gene-Eden, how would you describe your ability exercise (sports, climb stairs, walk several blocks . . . ), on a scale from 1 to 7?

| | | | | | | | | |
|---|---|---|---|---|---|---|---|---|
| Completely Unable | 1 | 2 | 3 | 4 | 5 | 6 | 7 | Completely Able |

C.4 Before taking Gene-Eden, how would you describe your ability to do house work (cleaning, laundry, cooking, shopping) on a scale from 1 to 7?

| | | | | | | | | |
|---|---|---|---|---|---|---|---|---|
| Completely Unable | 1 | 2 | 3 | 4 | 5 | 6 | 7 | Completely Able |

Since taking Gene-Eden, how would you describe your ability to do house work (cleaning, laundry, cooking, shopping), on a scale from 1 to 7?

| | | | | | | | | |
|---|---|---|---|---|---|---|---|---|
| Completely Unable | 1 | 2 | 3 | 4 | 5 | 6 | 7 | Completely Able |

C.5 Before taking Gene-Eden, how would you describe your ability to wash and dress yourself on a scale from 1 to 7?

| | | | | | | | | |
|---|---|---|---|---|---|---|---|---|
| Completely Unable | 1 | 2 | 3 | 4 | 5 | 6 | 7 | Completely Able |

Since taking Gene-Eden, how would you describe your ability to wash and dress yourself, on a scale from 1 to 7?

| | | | | | | | | |
|---|---|---|---|---|---|---|---|---|
| Completely Unable | 1 | 2 | 3 | 4 | 5 | 6 | 7 | Completely Able |

C.6 Before taking Gene-Eden, how would you describe your ability to get out of bed in the morning on a scale from 1 to 7?

| | | | | | | | | |
|---|---|---|---|---|---|---|---|---|
| Completely Unable | 1 | 2 | 3 | 4 | 5 | 6 | 7 | Completely Able |

Since taking Gene-Eden, how would you describe your ability to get out of bed, on a scale from 1 to 7?

| | | | | | | | | |
|---|---|---|---|---|---|---|---|---|
| Completely Unable | 1 | 2 | 3 | 4 | 5 | 6 | 7 | Completely Able |

D. Energy/Fatigue

Now we are moving to questions about your energy/fatigue.

D.1 Before taking Gene-Eden, how would you describe your level of energy, liveliness, vitality on a scale from 1 to 7?

| | | | | | | | | |
|---|---|---|---|---|---|---|---|---|
| No Energy at All | 1 | 2 | 3 | 4 | 5 | 6 | 7 | Full of Energy |

Since taking Gene-Eden, how would you describe your level of energy, liveliness, vitality, on a scale from 1 to 7?

| | | | | | | | | |
|---|---|---|---|---|---|---|---|---|
| No Energy at All | 1 | 2 | 3 | 4 | 5 | 6 | 7 | Full of Energy |

D.2 Before taking Gene-Eden, how easy or fast did you get tired during the day, on a scale from 1 to 7?

| | | | | | | | | |
|---|---|---|---|---|---|---|---|---|
| Very Easy | 1 | 2 | 3 | 4 | 5 | 6 | 7 | Never Got Tired |

Since taking Gene-Eden, how easy or fast do you get tired during the day, on a scale from 1 to 7?

| | | | | | | | | |
|---|---|---|---|---|---|---|---|---|
| Very Easy | 1 | 2 | 3 | 4 | 5 | 6 | 7 | Never Got Tired |

D.3 Before taking Gene-Eden, how well did you sleep, on a scale from 1 to 7?

| | | | | | | | | |
|---|---|---|---|---|---|---|---|---|
| Couldn't Sleep | 1 | 2 | 3 | 4 | 5 | 6 | 7 | I Slept Very Well |

Since taking Gene-Eden, how well do you sleep, on a scale from 1 to 7?

| | | | | | | | | |
|---|---|---|---|---|---|---|---|---|
| Couldn't Sleep | 1 | 2 | 3 | 4 | 5 | 6 | 7 | I Slept Very Well |

D.4 Before taking Gene-Eden, how stiff were you during the day, on a scale from 1 to 7?

| | | | | | | | | |
|---|---|---|---|---|---|---|---|---|
| Very Stiff | 1 | 2 | 3 | 4 | 5 | 6 | 7 | Very Flexible |

Since taking Gene-Eden, how stiff were you during the day, on a scale from 1 to 7?

| | | | | | | | | |
|---|---|---|---|---|---|---|---|---|
| Very Stiff | 1 | 2 | 3 | 4 | 5 | 6 | 7 | Very Flexible |

D.5 Before taking Gene-Eden, how difficult was it to move, on a scale from 1 to 7?

| | | | | | | | | |
|---|---|---|---|---|---|---|---|---|
| Suffered Very Much | 1 | 2 | 3 | 4 | 5 | 6 | 7 | Not a Problem |

Since taking Gene-Eden, how difficult was it to move, on a scale from 1 to 7?

| | | | | | | | | |
|---|---|---|---|---|---|---|---|---|
| Very Much | 1 | 2 | 3 | 4 | 5 | 6 | 7 | Not At All |

E. Mental Tasks

Now I will ask you questions about your thinking

E.1 Before taking Gene-Eden, how would you describe your attention span (concentration, clear thinking), on a scale from 1 to 7?

| | | | | | | | | |
|---|---|---|---|---|---|---|---|---|
| Very Poor | 1 | 2 | 3 | 4 | 5 | 6 | 7 | Very Strong |

Since taking Gene-Eden, how would you describe your attention span, on a scale from 1 to 7?

| | | | | | | | | |
|---|---|---|---|---|---|---|---|---|
| Very Poor | 1 | 2 | 3 | 4 | 5 | 6 | 7 | Very Strong |

E.2 Before taking Gene-Eden, how hard was it to form thoughts or make decisions, on a scale from 1 to 7?

| | | | | | | | | |
|---|---|---|---|---|---|---|---|---|
| Very hard | 1 | 2 | 3 | 4 | 5 | 6 | 7 | Very Easy |

Since taking Gene-Eden, how hard is it to form thoughts or make decisions, on a scale from 1 to 7?

| | | | | | | | | |
|---|---|---|---|---|---|---|---|---|
| Very hard | 1 | 2 | 3 | 4 | 5 | 6 | 7 | Very Easy |

E.3 Before taking Gene-Eden, how hard was it to concentrate, on a scale from 1 to 7?

| | | | | | | | | |
|---|---|---|---|---|---|---|---|---|
| Very hard | 1 | 2 | 3 | 4 | 5 | 6 | 7 | Very Easy |

Since taking Gene-Eden, how hard is it to concentrate, on a scale from 1 to 7?

| | | | | | | | | |
|---|---|---|---|---|---|---|---|---|
| Very hard | 1 | 2 | 3 | 4 | 5 | 6 | 7 | Very Easy |

E.4 Before taking Gene-Eden, how fast was your thinking, on a scale from 1 to 7?

| | | | | | | | | |
|---|---|---|---|---|---|---|---|---|
| Very Slow | 1 | 2 | 3 | 4 | 5 | 6 | 7 | Very Fast |

Since taking Gene-Eden, how fast is your thinking, on a scale from 1 to 7?

| | | | | | | | | |
|---|---|---|---|---|---|---|---|---|
| Very Slow | 1 | 2 | 3 | 4 | 5 | 6 | 7 | Very Fast |

E.5 Before taking Gene-Eden, how hard was it to remember things, on a scale from 1 to 7?

| | | | | | | | | |
|---|---|---|---|---|---|---|---|---|
| Very hard | 1 | 2 | 3 | 4 | 5 | 6 | 7 | Very easy |

Since taking Gene-Eden, how hard is it to remember things, on a scale from 1 to 7?

| | | | | | | | | |
|---|---|---|---|---|---|---|---|---|
| Very hard | 1 | 2 | 3 | 4 | 5 | 6 | 7 | Very easy |

E.6 Before taking Gene-Eden, how often did you get confused, on a scale from 1 to 7?

| | | | | | | | | |
|---|---|---|---|---|---|---|---|---|
| Very Often | 1 | 2 | 3 | 4 | 5 | 6 | 7 | Not At All |

Since taking Gene-Eden, how often do you get confused, on a scale from 1 to 7?

| | | | | | | | | |
|---|---|---|---|---|---|---|---|---|
| Very Often | 1 | 2 | 3 | 4 | 5 | 6 | 7 | Not At All |

The NotCiq questionnaire is a patient reported outcome (PRO) instrument. The purpose of a PRO instrument is to capture the patient's experience. It should be a reliable measure that can support the claimed concept (FDA, 2006). As such, the primary endpoint should be defined. The main endpoint in this study was level of fatigue. Meaning, the objective of the study was to measure the effect of the treatment with Gene-Eden-VIR on the level of fatigue as it is reported by the treated participants.

To develop the questionnaire, several questionnaires found in the literature that measured similar constructs were analyzed. The analysis considered the purpose of study, the research question, the scale and response format, the phrasing of tested items, and the process of determining its reliability and validity. At the end, a five section questionnaire was created. One section was designed to measure the changes in general health. A second section centered on the changes in the severity, duration, and frequency of the symptoms during a viral infection. A third section centered on changes in the ability to perform physical tasks, a fourth on energy/fatigue, and a fifth on the ability to perform mental tasks. The general health section included 5 questions. The section explored areas such as current disease and the change in general health. The symptoms section included 5 questions, two questions on severity, two questions on duration, and one question on the frequency of symptoms. The physical section included 5 questions, the energy/fatigue section included 5 questions, and the mental section included 6 questions. NotCiq included both open and closed-ended questions. The answers to the closed-ended questions were on a scale of 1 to 7, where “1” corresponded to “Poor health,” “Very Severe,” “Extremely Interfered,” “All the time,” “No relief,” or “Frequently appeared,” and 7 to “Unnoticeable,” “Not At All,” “Constant Relief,” or “Never Appeared,” etc.

The study collected the answers to the NotCiq instrument by phone interviews. The study used two independent companies that specialized in outbound call services for performing the interviewers, one company from the US and one from Israel. The interviewers were blinded to the objective of the study. All interviews were recorded.

The instrument was pre-tested on a small sample of Gene-Eden-VIR users to evaluate both the sensitivity and clarity of the questions.

Population

The study randomly selected participants from the Gene-Eden VIR customer database that includes all Gene-Eden-VIR current and past customers. The traditional response rate to phone interviews is 10-15%. Since the study was aiming to collect 100-150 interviews, the study used a computerized system to randomly create a call list of a 1000 customers. Out of the customers who agreed to participate, 95 Americans ages 20 to 66 reported suffering from some aspect of fatigue.

Since the objective of the study was to test the effect of Gene-Eden-VIR on different aspects of fatigue, the study excluded participants who reported no decrease in their energy levels, no decrease in their ability to perform physical tasks, or no decrease in their ability to perform mental tasks. That is, it excluded participants who reported a 7 point score on the pre-treatment question in these sections. Such score indicates that the participant does not suffer from the specific aspect of fatigue indicated by the question regardless of the treatment the participant actually received. This exclusion still preserved the intention to treat (ITT) principle.

Many of participants reported to be suffering from various conditions, including: viral infections, chronic fatigue syndrome (CFS), fibromyalgia, arthritis, diabetes, heart disease, obesity, chronic nervous syndromes, cancer, warts, fever blisters, thyroiditis, fatigue, stroke, and high blood pressure.

The study considered participants who stopped taking Gene-Eden-VIR for a month or more before data collection as past users. All other participants were considered as present users.

Controls

The Gene-Eden-VIR post marketing study includes a pre-treatment concurrent control and an historical control. To create an historical control, the study divided the original test group into two subgroups, present users and past users. Generally, an historical control is a separate group. However, since the study did not have a separate group of non-users, it used the past users as a proxy for an historical control.

Statistical Analysis

We tested the statistical difference between the score of ‘pre-treatment’, which is the numeric answer each participant used to describe his symptoms before the treatment started, to the score of ‘post-treatment’, which is the numeric answer each participant used to describe his symptoms after the treatment was completed. The delta (A), that is, the difference in scores between the answers to the pre-treatment and post-treatment question was also calculated. Then, the statistical difference between the deltas was tested. These tests were performed in a then-test model for both present and past users.

Statistical analysis was performed using a two-sample assuming unequal variances t-test.

The research defined the primary endpoint as a statistically significant increase in the score from pre-treatment to post-treatment on the raw answers and on the deltas.

Results

Response to Treatment

The participants reported no side effects from Gene-Eden-VIR.

Treatment with Gene-Eden-VIR decreased the fatigue level in 69 out of the 95 (73%) participants. Specifically, the treatment showed a statistically significant decrease in fatigue level as indicated by questions D1-D5, C1-C4, and C6, and E1-E5 (Table 2, p=0.046 to p<0.001). These clinical results prove that treatment with Gene-Eden-VIR decreases fatigue levels. Treatment also improved general health.

TABLE 2
No-treatment vs. treatment by question

| | No. of | No- | | |
|---|---|---|---|---|
| | Obser- | Treatment | Treatment | |
| Question | vations | Score | Score | P Value |
| A5-General health | 75 | 4.73 | 5.61 | <0.001 |
| D1-Level of energy | 76 | 4.66 | 5.80 | <0.001 |
| D2-How fast did you | 76 | 4.21 | 5.42 | <0.001 |
| get tired | | | | |
| D3-Ability to sleep | 68 | 4.41 | 5.21 | <0.001 |
| D4-Stiffness | 55 | 4.51 | 5.15 | 0.007 |
| D5-Ability to move | 36 | 4.97 | 5.47 | 0.046 |
| C1-How strong are you | 74 | 4.80 | 5.53 | <0.001 |
| C2-Ability to do your job | 44 | 4.95 | 5.55 | 0.038 |
| C3-Ability to exercise | 50 | 4.71 | 5.52 | 0.001 |
| C4-Ability to do | 46 | 4.89 | 5.53 | 0.018 |
| housework | | | | |
| C5-Ability to wash | 13 | 4.69 | 5.77 | 0.124 |
| and dress | | | | |
| C6-Ability to get | 54 | 4.45 | 5.38 | <0.001 |
| out of bed | | | | |
| E1-Attention span | 59 | 4.88 | 5.53 | 0.001 |
| E2-Ability to form | 47 | 5.09 | 5.62 | 0.012 |
| thoughts | | | | |
| E3-Ability to concentrate | 52 | 4.81 | 5.52 | 0.002 |
| E4-Ability to think fast | 55 | 5.25 | 5.64 | 0.017 |
| E5-Ability to remember | 61 | 4.95 | 5.49 | 0.003 |
| E6-Being confused | 43 | 5.02 | 5.49 | 0.056 |

To test the dose effect of Gene-Eden-VIR, the study compared the change (A) from no-treatment to treatment for participants who took 2 capsule per day (n=193) and participants who took 4 capsules per day (n=75). The results showed that those who took 4 capsules per day experienced a larger decrease in their fatigue level compared to those who took 2 capsule per day (Table 3, p<0.018). The number of participants taking 1 and 3 capsules was too small for statistical analysis.

TABLE 3
Dose: one capsule per day vs. two capsule per day

| | | Change (Δ) from No- | |
|---|---|---|---|
| | No. of | Treatment to | |
| Dose of treatment | Observations | Treatment | P Value |
| 2 capsule/day | 193 | 0.75 | 0.018 |
| 4 capsules/day | 75 | 1.11 | |

To test the effect of duration of treatment, the study compared the change (A) from no-treatment to treatment for participants who took Gene-Eden-VIR for less then three months to those who took Gene-Eden-VIR for three months or more. The results showed that those who took Gene-Eden-VIR for the longer period experienced a larger decrease in their fatigue level (Table 4, p<0.003).

TABLE 4
Duration: less than 3 month vs. more than 3 month

| | | Change (Δ) from | |
|---|---|---|---|
| Duration of | No. of | No-Treatment to | |
| treatment | Observations | Treatment | P Value |
| <3 months | 222 | 0.78 | 0.003 |
| ≧3 months | 89 | 1.26 | |

To test for a possible interviewer bias, the study compared the change (A) from no-treatment to treatment collected by the American and the Israeli call centers. The results showed no difference between the answers (Table 5, p=0.238). This means that although the centers included different interviewers from different cultures working at different times of day, Americans working during the day and Israelis working during the night, the answers were similar. Hence, the results show no interviewer bias.

TABLE 5
USA vs. Israel call centers

| | | Change (Δ) from No- | |
|---|---|---|---|
| | No. of | Treatment to | |
| Call center | Observations | Treatment | P Value |
| USA | 181 | 0.88 | 0.238 |
| Israel | 130 | 0.98 | |

To test for possible selection bias, the study compared the answers to the no-treatment questions collected from past and present users of Gene-Eden-VIR. The study also compared the answers to the treatment questions between the two groups. In both cases, there was no difference between the answers (Table 6, p=0.405 for no-treatment, p=0.331 for treatment). This means that present users experience the same decrease in the fatigue level as past users, and therefore, the results showed no selection bias.

TABLE 6
Past vs. present users

| | Past | | | | |
|---|---|---|---|---|---|
| | No. of | | Present | | |
| | Obser- | | No. of | | P |
| Variable | vations | Score | Observations | Score | Value |
| No | 87 | 4.46 | 218 | 4.50 | 0.405 |
| treatment | | | | | |
| Treatment | 87 | 5.46 | 218 | 5.38 | 0.331 |

According to the literature on ‘response shifts’, participants may alter their internal standards, values, or conceptualization of their quality of life when experiencing changes in health states. These response shifts can affect or distort the reported scores and undermine the credibility of the observed medical or psychosocial effects. Many studies report that after participants experience an improvement in their health, a then-test tends to show a decrease in the initial assessment of the original level of well being.

To test for a possible response shift, the study compared the answers to the no-treatment question at different time points. The first point was 4 weeks from commencement of treatment. At this point, there is little to no increase in fatigue level (see table 4), and therefore can serve as a substitute for the pre-test state. The other time points were 12, 26, and 52 weeks from commencement of treatment. The results showed that in all three cases, there is no change in the scores of the no-treatment questions (Table 7). Hence, the results indicate that this study showed no response shift.

TABLE 7
No-treatment scores overtime

| | No. of | | | |
|---|---|---|---|---|
| No. of weeks | Observations | Score | Test | P Value |
| 4 weeks | 87 | 4.47 | | |
| 12 weeks | 37 | 4.41 | 4 vs. 12 weeks | 0.387 |
| 26 weeks | 26 | 4.65 | 4 vs. 26 weeks | 0.289 |
| 52 weeks | 26 | 4.65 | 4 vs. 52 weeks | 0.262 |

An issue unique to natural products is a concern about the therapeutic consistency of marketed products. See discussion on this issue in the FDA guidelines for botanical New Drug Applications (NDA) (FDA 2004). To test the therapeutic consistency of Gene-Eden-VIR, the study compared the two batches used by the participants. The capsules in these batches were produced at two different manufacturing sites, and completed about 10 months apart. The results showed that the decrease in fatigue level is the same for the two batches (Table 8, p=0.319). Hence, the results indicated that, although Gene-Eden-VIR is a natural product, its formula has therapeutic consistency.

TABLE 8
Batch: Batch 1 vs. Batch 2

| | | Change (Δ) from No- | |
|---|---|---|---|
| | No. of | Treatment to | |
| Batch | Observations | Treatment | P Value |
| 1 | 177 | 0.95 | 0.319 |
| 2 | 129 | 0.88 | |

Summary

According to the FDA website, the “FDA uses postmarketing study commitments to gather additional information about a product's safety, efficacy, or optimal use.” This post marketing study was used for the same objective, that is, to test the efficacy, safety, and optimal use of Gene-Eden-VIR for decreasing fatigue levels. The study showed that that individuals suffering from excess fatigue reported a safe decrease in their fatigue levels following treatment with Gene-Eden-VIR.

The results are consistent. The study showed a statistical significant decrease in fatigue levels. The results also showed a duration effect. Participants treated for three months or more reported a larger decrease in their fatigue levels compared to those treated for less then three months.

The results are robust. They showed no interviewer bias, no selection bias, and therapeutic consistency of the Gene-Eden-VIR formula under varying manufacturing conditions. The results also showed no response shift.

This post marketing clinical study does not include a placebo control, that is, it is not a double blinded study. Placebo controlled studies are the gold standard in medical research in pre marketing clinical studies. However, except in rare cases, post marketing studies do not use placebo controls. They use other controls recommended by the FDA.

According to the FDA Guidance to the Industry (FDA 2001):

“Control groups have one major purpose: to allow discrimination of patient outcomes (for example, changes in symptoms, signs, or other morbidity) caused by the test treatment from outcomes caused by other factors, such as the natural progression of the disease, observer or patient expectations, or other treatment. The control group experience tells us what would have happened to patients if they had not received the test treatment or if they had received a different treatment known to be effective.”

The FDA guidance mentions five types of controls used in both pre marketing and post marketing studies: (1) Placebo Concurrent Control, (2) Pre-treatment Concurrent Control, (3) Dose-response Concurrent Control, (4) Active (Positive) Concurrent Control, (5) External Control (Including Historical Control). The External Control “can be a group of patients treated at an earlier time (historical control).”

No-treatment Concurrent Control, a Dose-response Concurrent Control, and an Historical Control. These are three out of five controls accepted by the FDA in their drug approval process. The use of these controls indicates that the Gene-Eden-VIR study is a well-controlled clinical study.

In a post marketing study one must consider the effect of cognitive dissonance, that is, the condition where people are holding two conflicting beliefs. Under cognitive dissonance, people have a motivational drive to reduce the conflict by altering these beliefs, adding new ones to create a consistent belief system, or reducing the importance of one of the conflicting elements. In this study one can expect the existence of cognitive dissonance that may result from guilt, commitment, or the need to justify the purchasing and using of the product to oneself by believing that the product actually works. It is possible that the past users are less likely to experience such dissonance since they no longer use the product, and hence, feel no commitment to report a positive effect. Moreover, past users might have the opposite dissonance, that is, a need to justify their decision to stop using the product, and hence, report no effect or even a negative effect. The results showed that past and present users report a similar decrease in their symptoms. This similarity indicates that cognitive dissonance was not involved in determining the participants' reports.

All participants who started the study completed it; therefore the study has no follow-up bias.

It should be noted that although the study tested for some biases, others are still possible, for instance, the non-responsive bias.

The results are not likely to be a placebo effect. The current predominant and well-proven theories on the placebo effect suggest that its main mechanisms are conditioned reflexes and patient expectations (Breidert and Hofbauer 2009). The Gene-Eden-VIR product literature, website, or any other written or oral public communications, did not mention the possibility of a change in fatigue levels, energy levels, or the ability to perform physical tasks. Hence, the participants in this study could not have been primed for, or expect the reported effects. This lack of conditioned reflexes and patient expectations minimizes the possibility of a placebo effect, and supports the possibility of a physiological effect.

This study relies on patient reported outcomes (PROs). Past studies showed that PROs had a significant role in the development and evaluation of new medicines (Willke 2004). From the years 1997 to 2002, the FDA approved 23 new drugs based on PRO endpoints only. They include six anti-migraine products (Amerge®, Axert®), several anti-epileptics (Gabitril®, Keppra®), and a variety of other therapy classes (Tamiflu®, Relenza®) (Willke 2004).

Consider the FDA's opinion on the issue. According to the FDA 2006 (underline added): “A PRO is a measurement of any aspect of a patient's health status that comes directly from the patient (i.e., without the interpretation of the patient's responses by a physician or anyone else). In clinical trials, a PRO instrument can be used to measure the impact of an intervention on one or more aspects of patients' health status, hereafter referred to as PRO concepts, ranging from the purely symptomatic (response of a headache) to more complex concepts (e.g., ability to carry out activities of daily living), to extremely complex concepts such as quality of life, which is widely understood to be a multidomain concept with physical, psychological, and social components. Data generated by a PRO instrument can provide evidence of a treatment benefit from the patient perspective.

Generally, findings measured by PRO instruments may be used to support claims in approved product labeling if the claims are derived from adequate and well-controlled investigations that use PRO instruments that reliably and validly measure the specific concepts at issue.

2.1 Why use patient-reported outcome instruments in medical product development? PRO instruments are included in clinical trials for new medical products because (1) some treatment effects are known only to the patient; (2) there is a desire to know the patient perspective about the effectiveness of a treatment; or (3) systematic assessment of the patient's perspective may provide valuable information that can be lost when that perspective is filtered through a clinician's evaluation of the patient's response to clinical interview questions.”

PRO are also discussed in scientific papers. According to Demuro 2012 (underline added):

“Patient-Reported Outcome (PRO) use is particularly common for products developed to treat chronic, disabling conditions where the intention is not necessarily to cure but to ameliorate symptoms, facilitate functioning, or improve quality of life. PROs are the primary end points in clinical trials evaluating drug products for disease areas such as irritable bowel syndrome, migraine, and pain. PROs provide key supportive data in many other disease areas, such as insomnia, asthma, and psychiatric disorders. In oncology, PROs are commonly used to assess both treatment benefits and toxicity to fully evaluate the impact of treatment on health-related quality of life (HRQOL). PROs can also be used in clinical trials to assess treatment satisfaction, compliance, and caregiver burden.

Willke and colleagues [2] conducted a review of drug labels to understand the use of PROs compared with other trial end points. That research identified the inclusion of PROs as efficacy end points in approximately 30% of all labels reviewed between 1997 and 2002. In 2006, the Food and Drug Administration (FDA) released a draft guidance for use of PROs in clinical trials, followed by a final guidance in 2009, Guidance for Industry: Patient Reported Outcome Measures: Use in Medical Product Development to Support Labeling Claims, providing a blueprint for the use of PROs in clinical trials. The guidance documents were intended to influence the appropriate development, validation, and use of PRO measures to facilitate a positive regulatory review in support of label claims.

Gnanasakathy and colleagues built on the work previously conducted by Willke and colleagues and reported the frequency of PROs in recently approved drug labels. Specifically, these authors found that PRO claims were granted for approximately 24% of all labels reviewed between January 2006 and December 2010.”

According to the FDA, PROs are a valid and valuable source for measuring the efficacy of new drugs. PROs are reliable enough to warrant an approval of a label claim for a new drug. Obviously, if the FDA regards this source of data as valid and valuable, so should the medical community. And indeed, many major journals publish clinical study that relies on patient reported outcomes.

The size of the study group is a major concern in clinical studies. A group that is too small may fail to show a positive effect of the treatment. In addition, a small group could also misrepresent the diversity in the population. The standard principle for multivariate behavioral research is at least 10 patients at endpoint per dependent measure (Harvey and Keefe 2001). This study included one endpoint dependent measure (the change in fatigue levels from pre-treatment to post-treatment). This study population included 90 individuals. Hence, the size of the study group in this study is adequate.

One might also question the reliability of the participants recall due to the long duration of the time period under investigation (up to 54 weeks). This study used a ‘then-test’ method. This method, also known as the retrospective pre-test-post-test design method, asks participants at the post-test period to think back to the pre-test period and retrospectively rate their condition. The ‘response shift’ is defined as the difference between the ‘pre-test’ and the ‘then-test’ ratings. Currently, the response shift is a well documented and extensively research phenomenon (Schwartz 2006). According to the literature on ‘response shifts’, participants may alter their internal standards, values, or conceptualization of their quality of life when experiencing changes in health states. These response shifts can affect or distort the reported scores and undermine the credibility of the observed medical or psychosocial effects. Many studies reported that after participants experience an improvement in their health, a then-test tends to show a decrease in the initial assessment of the original level of well being.

Since this clinical study uses the ‘then-test’ method, the study tested for a possible response shift by comparing the answers to the pre-treatment question at 4, 12, 26, and 52 weeks. The results showed no statistically significant difference in the pre-treatment scores over time. Hence, the results in this study showed no response shift.

To conclude:

Currently there no systemic treatments approved for decreasing fatigue levels for the general population. This clinical study proved that the natural product Gene-Eden-VIR safely and effectively decreases fatigue levels, and more specifically, increases energy levels, and increases the ability to perform physical and mental tasks in treated individuals.

### REFERENCES

1. Huskisson E, Maggini S, Ruf M. The role of vitamins and minerals in energy metabolism and well-being. J Int Med Res. 2007 May-June; 35(3):277-89

2. Havard C W. Lassitude. Br Med J (Clin Res Ed). 1985 Apr. 20;290(6476):1161-2

3. Cho H J, Bower J E, Kiefe C I, Seeman T E, Irwin M R. Early life stress and inflammatory mechanisms of fatigue in the Coronary Artery Risk Development in Young Adults (CARDIA) study. Brain Behav Immun. 2012 Apr. 25

4. Appels A, Mulder P. Fatigue and heart disease. The association between ‘vital exhaustion’ and past, present and future coronary heart disease. J Psychosom Res. 1989;33(6):727-38

5. Judge R, Plewes J M, Kumar V, Koke S C, Kopp J B. Changes in energy during treatment of depression: an analysis of fluoxetine in double-blind, placebo-controlled trials. J Clin Psychopharmacol. 2000 December; 20(6):666-72

6. Theobald D E. Cancer pain, fatigue, distress, and insomnia in cancer patients. Clin Cornerstone. 2004; 6 Suppl 1D:S15-21

7. Lakhan S E, Vieira K F. Nutritional and herbal supplements for anxiety and anxiety-related disorders: systematic review. Nutr J. 2010 Oct. 7; 9:42

8. Barnes P M, Bloom B, Nahin R L. Complementary and alternative medicine use among adults and children: United States, 2007. Natl Health Stat Report. 2008 Dec. 10; (12):1-23

9. Johnson P J, Ward A, Knutson L, Sendelbach S. Personal use of complementary and alternative medicine (CAM) by U.S. health care workers. Health Serv Res. 2012 February; 47(1 Pt 1):211-27

10. US Department of Health and Human Services. Food and Drug Administration. Center for Drug Evaluation and Research (CDER). Guidance for industry. Botanical drug products. 2004

11. US Department of Health and Human Services. Food and Drug Administration. Center for Drug Evaluation and Research (CDER). Guidance for Industry, E 10 Choice of Control Group and Related Issues in Clinical Trials. May 2001

12. Breidert M, Hofbauer K. Placebo: Misunderstandings and Prejudices. Dtsch Arztebl Int. 2009 November; 106(46):751-5

13. Willke R J, Burke L B, Erickson P. Measuring treatment impact: a review of patient-reported outcomes and other efficacy endpoints in approved product labels. Control Clin Trials. 2004 December; 25(6):535-52

14. U.S. Department of Health and Human Services FDA Center for Drug Evaluation and Research. Guidance for industry: patient-reported outcome measures: use in medical product development to support labeling claims: draft guidance. Health Qual Life Outcomes. 2006 Oct. 11; 4:79.

15. Demuro C, Clark M, Mordin M, Fehnel S, Copley-Merriman C, Gnanasakthy A. Reasons for Rejection of Patient-Reported Outcome Label Claims: A Compilation Based on a Review of Patient-Reported Outcome Use among New Molecular Entities and Biologic License Applications, 2006-2010. Value Health. 2012 May; 15(3):443-8

16. Harvey P D, Keefe R S. Studies of cognitive change in patients with schizophrenia following novel antipsychotic treatment. Am J Psychiatry. 2001 February; 158(2):176-84.

17. Schwartz C E, Bode R, Repucci N, Becker J, Sprangers M A, Fayers P M. The clinical significance of adaptation to changing health: a meta-analysis of response shift. Qual Life Res. 2006 November; 15(9):1533-50.

We claim the following:

1. A method for treating an animal or human subject, the method comprising the steps of:
a. Selecting an agent, wherein the agent consists of a green tea extract, quercetin, licorice extract, cinnamomum extract, and selenium;
b. Administering the agent to the subject to decrease fatigue levels in said subject.

2. The method in claim 1, wherein said decrease in fatigue levels is indicated by an increase in energy levels.

3. The method in claim 1, wherein said decrease in fatigue levels is indicated by an increase in the ability to perform physical tasks.

4. The method in claim 1, wherein said decrease in fatigue levels is indicated by an increase in the ability to perform mental tasks.

5. The method in claim 1, wherein said subject is suffering from one or more of the following conditions: infection, malignancy, cardiac diseases, inflammation, medications, psychiatric disorders, neurological disorders, endocrine disorders, rheumatologic disorders, genetic disorders, metabolic disorders, electrolyte disorders, chronic fatigue syndrome (CFS), fibromyalgia, arthritis, diabetes, heart disease, obesity, chronic nervous syndromes, cancer, thyroiditis, stroke, multiple sclerosis, hypertension, traumatic brain injury, other neurologic conditions, cardiovascular disease, systemic lupus erythematosus, depression, sleeping disorders, psychological disorders, fear, stress, anxiety, and obsessive and compulsive thoughts.

6. The method in claim 1, wherein fatigue is indicated by a symptom selected from the groups consisting of a decrease in energy levels, a decrease in ability to perform physical tasks, and/or decrease in ability to perform mental tasks.
